(19) United States
(12) Patent Application Publication
(10) Pub. No.: US 2006/0271011 A1
(43) Pub. Date: Nov. 30, 2006

(54) Methods and apparatus for indicating when a disposable component of a drug delivery system needs to be replaced

(76) Inventors: Bradley Dean Mock, Greenwich, CT (US); Nicholas Arthur Kiratsous, Greenwich, CT (US)

Correspondence Address: Gordon & Jacobson, P.C., Suite 407, 60 Long Ridge Road, Stamford, CT, 06902, US

(21) Appl. No.: 11/137,045

(22) Filed: May 25, 2005

(51) Int. Cl. A61M 31/00

(52) U.S. Cl. 604/500; 604/111

(57) ABSTRACT

Methods for indicating when a disposable component of a drug delivery system needs to be replaced include providing a timer which is associated with the drug delivery device or the disposable component thereof. The timer may be static or dynamic and may count up or down. The timer may be reusable or disposable, and may be attached to the drug delivery device, or to the disposable component or to a case in which the drug delivery device is kept.

### BACKGROUND OF THE INVENTION

1. Field of the Invention

This invention relates broadly to drug delivery systems. More particularly, this invention relates to methods and apparatus for indicating when a disposable component of a drug delivery system needs to be replaced.

2. State of the Art

A recent breakthrough in drug delivery systems is the “dry powder pulmonary drug delivery system”. With these systems, a patient can inhale a drug which previously required injection with a hypodermic needle. This new drug delivery system presents a great benefit to diabetes patients who can now inhale insulin rather than inject it with a needle.

Prior art FIG. 1 illustrates a typical dry powder pulmonary drug delivery system 10, such as available from Nektar Therapeutics of San Carlos, Calif. The system 10 includes a base unit 12 and a removable chamber 14. The base unit 12 includes a pump lever 16, a trigger or fire button 18, a blister slot 22, and a transjector 24. The chamber 14 includes a cap 26 bearing an inhaler nozzle 28.

A single dose (e.g. 1-10 mg) of a dry powder drug is provided in a blister 30 which is inserted into the blister slot 22 of the base 12. The chamber 14 is normally attached to the base and slides down and covers it, The patient lifts the base up to the position shown in FIG. 2 to use the system. The chamber is removable as shown in FIG. 1 for cleaning. The patient lifts the lever 16 and presses it down to prime the device with air. The patient then presses the trigger 18. The device rapidly disperses the powder contained in the blister 30 via a small burst (e.g. 10 mg) of compressed air which discharges the powder through small jets in the transjector 24 into the chamber 14. The dispersal is accomplished in less than 80 milliseconds. The patient then places the nozzle 28 in his/her mouth as shown in prior art FIG. 2 and inhales the particles. More than 90% of the particles inhaled from the chamber 14 via the nozzle 28 have diameters less than 5 microns and a high fraction of the starting mass in the blister is inhaled through the nozzle 28.

Manufacturers of these devices recommend that the transjector be replaced periodically. The exact time when the transjector needs to be replaced will vary depending on the drug and the number and volume of doses dispensed. However, in order to simplify prescription information, it may be recommended that all patients replace the transjector at the end of a period of time, e.g. two weeks, regardless of the number of uses. This poses a challenge to the patients, particularly elderly patients who may have difficulty remembering to replace the transjector in a timely manner.

### SUMMARY OF THE INVENTION

It is therefore an object of the invention to provide methods for indicating when a transjector needs to be replaced.

It is another object of the invention to provide apparatus for indicating when a transjector needs to be replaced.

It is a further object of the invention to provide methods and apparatus for indicating when a disposable component of a drug delivery system needs to be replaced.

In accord with these objects, which will be discussed in detail below, the methods of the invention include providing a timer which is associated with the drug delivery device or the disposable component thereof. The timer may be static or dynamic and may count up or down. It may be reusable or disposable. The timer may be attached to the drug delivery device or to a case in which the drug delivery device is kept.

Exemplary timer apparatus according to the invention include an adhesive strip bearing a chemical which causes it to change color or display previously invisible text after it has been exposed to air for a predetermined period, an adhesive calendar which the patient marks to indicate the date on which the component needs to be replaced or the date when it was installed, a windowed disk which is static mounted over a circle of numbers to indicate the start and/or stop dates, a mechanical clock, an electric clock, a solid state timer with an LED, etc. All of the timers preferably have some means for attaching to either the drug delivery device or to the disposable component or to a case in which the drug delivery device is kept. The disposable timers preferably have some means for detaching from the drug delivery device or to the disposable component or to a case in which the drug delivery device is kept.

Additional objects and advantages of the invention will become apparent to those skilled in the art upon reference to the detailed description taken in conjunction with the provided figures.

### BRIEF DESCRIPTION OF THE DRAWINGS

FIG. 1 is an exploded perspective view of a prior art drug delivery device;

FIG. 2 is a schematic view of the device in use;

FIG. 3 is a schematic view of a first embodiment of a timer according to the invention;

FIG. 4 is a schematic view of a second embodiment of a timer according to the invention;

FIG. 5 is a schematic view of a third embodiment of a timer according to the invention;

FIG. 6 is a schematic view of a fourth embodiment of a timer according to the invention;

FIG. 7 is a schematic circuit diagram of a fifth embodiment of a timer according to the invention; and

FIG. 8 is a schematic view of the fifth embodiment in a package which is attachable to a drug delivery device or a drug delivery device case.

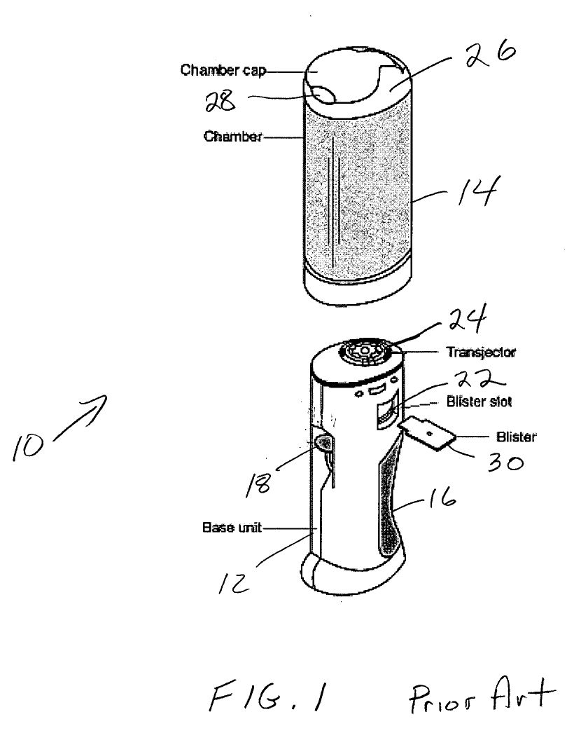

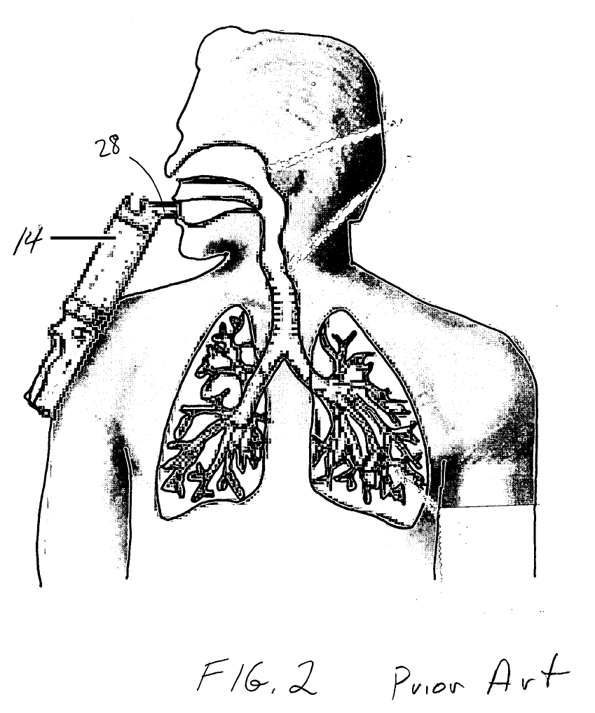

### DETAILED DESCRIPTION

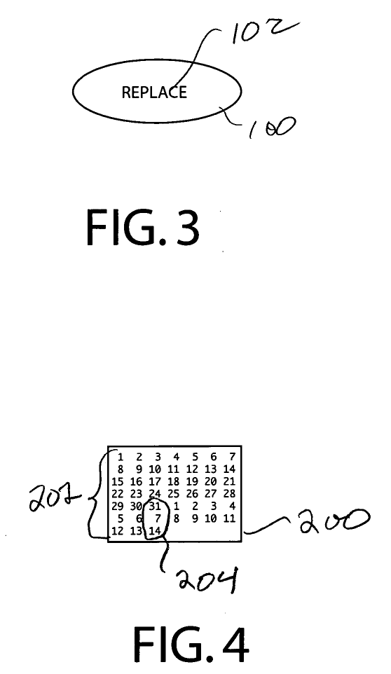

Turning now to FIG. 3, a timer 100 is shown. The timer 100 is preferably a self-adhesive sticker which contains indicia 102 (e.g. the word “replace”) which only becomes visible after the sticker has been exposed to air for a predetermined number of days, e.g. 14 days. Suitable technology for producing indicia which changes color after a predetermined period of time is disclosed in U.S. Pat. No. 5,364,132, the complete disclosure of which is hereby incorporated by reference herein and similar technology modified for color change after a relatively long period of time can be used to produce indicia 102. It will be appreciated that when using the timer of FIG. 3, the timer 100 must be packaged in an airtight container prior to use. Once the timer 100 is removed from the container, it should be attached to the drug delivery system or to a case containing the drug delivery system. The new disposable component should be put into service as soon as possible, lest the timer 100 indicate replacement earlier than necessary. An alternative embodiment using the same technology is to coat at least a patient-visible portion of the disposable component with the substance that changes color after a predetermined time. As yet another alternative, the disposable component can be manufactured from a color change substance that will change color after a predetermined period of time upon exposure to air.

FIG. 4 illustrates a second embodiment of a timer 200. The timer 200 is preferably a self-adhesive sticker bearing numeric indicia 202. The numeric indicia are preferably arranged in seven columns and seven rows. The indicia preferably consists of numerals 1 through 31 followed by numerals 1 through 14. The patient, upon installing the transjector 24 uses a marking device such as a pen to encircle the numeral representing the day of the month when the transjector is installed as well as the numeral two rows beneath it, or 14 days later. For example, the marking 204 illustrates that the transjector was installed on the thirty-first and needs to be replaced on the fourteenth.

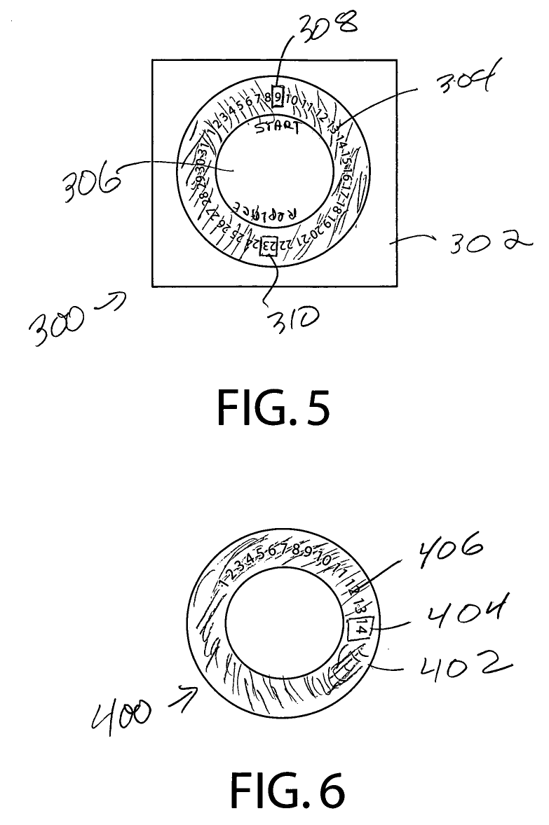

FIG. 5 illustrates an optionally re-usable timer 300 which may be attached to the drug delivery device or a case containing the device. The timer 300 includes a substrate 302 which is imprinted with numeric indicia 304 and an opaque vinyl overlay 306 having two windows 308, 310. The indicia includes the numerals 1 through 31 arranged in a circle. The windows 308, 310 on the overlay 306 are arranged so that when placed over the indicia they reveal two numerals which are spaced apart from each other by a numeric difference which equals the timer period, e.g. 14. The substrate 302 is coated with a material which allows the overlay to affix to it via static electricity. The same concept is used in the popular toy COLORFORMS®. Although the timer 300 is reusable, it is inexpensive enough that it could be disposable. The timer is used by arranging the overlay 306 so that one of the windows 308 exposes the numeral of the day of the month that the component is placed in service. The other window 310 automatically exposes the day of the month upon which the component is to be replaced. According to a preferred embodiment, the words “start” and “replace” are imprinted under the windows.

It is recognized that the embodiments shown in FIGS. 4 and 5 may be inaccurate by one to three days or may produce confusing results when the transjector is put into service in a month having fewer than thirty-one days. As such, these embodiments may not be preferred where accuracy of the timed interval is particularly important.

FIG. 6 illustrates a timer 400 which includes a casing 402 having a window 404. A rotating wheel 406 is mounted in the casing 402. The wheel is imprinted with indicia. As illustrated, the wheel is imprinted with the numerals 1 through 14. In other embodiments, the wheel could be imprinted with colors red, yellow, green or with the word “replace”. The wheel is caused to rotate by a clock mechanism which may be mechanical or electrical. As the wheel rotates, the indicia become visible through the window 404. In the illustrated embodiment, the visible numeric indicia changes once per day, thereby counting down from 14 to 1 or counting up from 1 to 14. When the timer ends its count, it is time to replace the disposable component. In alternate embodiments, a green stripe is visible through the window, then a yellow stripe and when a red stripe is visible, the count has ended and it is time to replace the component. In still another alternate embodiment, the last visible indicia is the word “replace”. The timer 400 may be reusable (with a setting stem to return the wheel to its original position) or may be disposable. In either case, the wheel is preferably provided with a lock to prevent it from turning until the component is put into service.

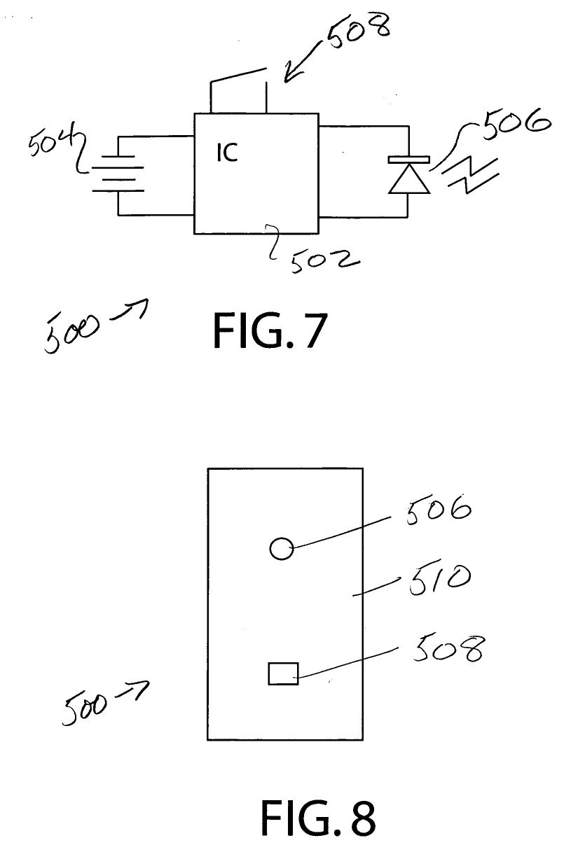

FIGS. 7 and 8 show an electronic timer 500. The timer includes an integrated circuit (IC) chip 502, a power source 504 coupled to the chip, and a visual indicator, e.g., LED 506, coupled to the chip. An actuation/reset pushbutton 508 is also provided. The components are mounted in a housing 510 which is provided with means for attaching it to a drug delivery device or a case containing a drug delivery device. When the reset button 508 is pressed, the timer counts a predetermined amount of time (e.g. 14 days) at the end of which the LED lights. Alternate programs may include that the LED blinks slowly while the timer is counting, more rapidly as it nears the end of the count and finally glows steadily at the end of the count. Multiple LEDs may also be arranged to spell out the word “replace”. Another alternate program is that the button 508 must be pressed and held for a number of seconds, thereby preventing accidental resetting of the timer. Still another alternate program would be for the LED to glow throughout the interval and go dark at the end. The timer 500 can be either reusable or disposable. A suitable IC is the LM 555 available from National Semiconductor. Other visual indicators, e.g., an LCD display, can also be used.

There have been described and illustrated herein several embodiments of methods and apparatus for indicating when a disposable component of a drug delivery system needs to be replaced. While particular embodiments of the invention have been described, it is not intended that the invention be limited thereto, as it is intended that the invention be as broad in scope as the art will allow and that the specification be read likewise. It will therefore be appreciated by those skilled in the art that yet other modifications could be made to the provided invention without deviating from its spirit and scope as claimed.

What is claimed is:

1. A method for indicating when a disposable component of a drug delivery device needs to be replaced, comprising:
providing a timer which, once set, is capable of indicating when the disposable component needs to be replaced,
associating the timer with the drug delivery device, or the disposable component thereof, or with a case containing the drug delivery device, and
setting the timer.

2. The method according to claim 1, wherein:
the timer is disposable, and
the step of associating includes attaching the timer to the delivery device.

3. The method according to claim 2, wherein:
the timer is activated by exposure to air.

4. The method according to claim 1, wherein:
the timer includes a self-adhesive sticker.

5. The method according to claim 1, wherein:
the timer is a sticker bearing numeric indicia, and
the step of setting includes marking the sticker with a marking device.

6. The method according to claim 1, wherein:
the timer includes a substrate bearing indicia, and
the step of setting the timer includes placing an overlay having at least one window over the substrate with less than all of the indicia visible through the window.

7. The method according to claim 6, wherein:
the overlay has two windows each exposing one of the indicia on the substrate.

8. The method according to claim 6, wherein:
the indicia include the numerals corresponding to the days of a month, and
the step of setting includes locating the window over the numeral corresponding to the day of the month when the component is put in service or over the day of the month when the component needs to be replaced.

9. The method according to claim 1, wherein:
the timer includes a motor driven wheel bearing indicia and a window which exposes only a portion of the indicia, the motor being either electrical or spring driven, and
the step of setting includes activating the motor.

10. The method according to claim 1,
wherein the timer includes an IC chip,
a power source, an activation button and a visual indicator coupled to the IC chip, and
the step of setting includes pressing the activation button.

11. The method according to claim 10, further comprising:
resetting the timer.

12. The method according to claim 1, further comprising:
providing the drug delivery device, wherein the drug delivery device is a dry powder pulmonary drug delivery system.

13. The method according to claim 1, wherein:
said step of associating includes attaching the timer to the drug delivery device.

14. A drug delivery system, comprising:
a patient operable reusable device for delivering doses of drug to the patient;
a patient disposable component used in conjunction with the reusable device and which contacts the drug during drug delivery; and
a timer which indicates when the patient disposable component needs to be replaced.

15. The system according to claim 14, wherein:
the timer indicates a predetermined period of time, regardless of the number of doses delivered.

16. The system according to claim 14, wherein:
the timer is activated by exposure to air.

17. The system according to claim 16, wherein:
the timer is a self-adhesive sticker.

18. The system according to claim 14, wherein:
the timer is a sticker bearing numeric indicia.

19. The system according to claim 14, wherein:
the timer includes a substrate bearing indicia and an overlay having at least one window such that when placed over the substrate, less than all of the indicia visible through the window.

20. The system according to claim 19, wherein:
the overlay has two windows each exposing one of the indicia on the substrate.

21. The system according to claim 19, wherein:
the indicia include the numerals corresponding to the days of a month.

22. The system according to claim 14, wherein:
the timer includes a motor driven wheel bearing indicia and a window which exposes only a portion of the indicia, the motor being either electrical or spring driven.

23. The system according to claim 14, wherein:
the timer includes an IC chip, with a power source, a reset button and a visual indicator coupled to the IC chip.

24. The component according to claim 14, wherein:
said drug delivery system is a dry powder pulmonary drug delivery system and said disposable component is a transjector.

25. A disposable component for use with a drug delivery system, comprising:
a timer coupled to or integrated into said disposable component, said timer indicating when said disposable component needs to be replaced in or on the drug delivery system.
